(19) United States
(12) Patent Application Publication
(10) Pub. No.: US 2012/0226188 A1
(43) Pub. Date: Sep. 6, 2012

(54) PARAMETRIC CONTROL OF VOLUME STREAMS

(75) Inventors: Matthias Schaefer, Frankfurt am Main (DE); Andreas Gille, Ascot Vale (AU)

(73) Assignee: SANOFI-AVENTIS DEUTSCHLAND GMBH, Frankfurt am Main (DE)

(21) Appl. No.: 13/391,735

(22) PCT Filed: Aug. 27, 2010
(86) PCT No.: PCT/EP10/62533

§ 371 Date: May 10, 2012

(30) Foreign Application Priority Data
Sep. 2, 2009 (EP) 09290669.2

(51) Int. Cl. A61M 5/168; A61B 5/00; A61B 10/02

(52) U.S. Cl. 600/562; 604/66; 600/573

(57) ABSTRACT

The invention relates to a medical device for infusion of an active ingredient or removal of a body tissue or body liquid comprising: a outlet device for infusion of an active ingredient to a body tissue; a receptacle for storing the active ingredient to which the outlet device is attachable; a sensor device for measuring a parameter which is related to a physical state in the body tissue; a drive device for effectuating at least an infusion of the active ingredient; characterized by a control unit functionally connected to the sensor device for receiving a sensor signal, which corresponds to the state of the body tissue, wherein the control unit is adapted for controlling a flow rate.

### CROSS REFERENCE TO RELATED APPLICATIONS

The present application is a U.S. National Phase Application pursuant to 35 U.S.C. §371 of International Application No. PCT/EP2010/062533 filed Aug. 27, 2010, which claims priority to European Patent Application No. 09290669.2 filed on Sep. 2, 2009. The entire disclosure contents of these applications are herewith incorporated by reference into the present application.

### FIELD OF INVENTION

The present invention relates to a medical device for infusion of an active ingredient or for removal of a body tissue. More particularly the present invention relates to a device for infusion of an active ingredient, being a medicament into the body tissue of a human body via a outlet device or to a device for removing a body tissue being for instance a biopsy sample of the human body.

### BACKGROUND

In the past the infusion of active ingredients or the removal of body tissue, both representing a transfer of volumes are, carried out automatically or manually when a patient undergoes a medical surgery. With the manual application via a syringe or a pen it is almost impossible to control the flow rate of the medicament applied to a patient. For those cases in which a certain dose per unit time has to be applied to a patient apparatuses have been developed for automated application of medicaments. In the known automatic methods a syringe is placed into an apparatus which acts on the syringe in order to deliver the active ingredient. Often, the delivery of a medicament by such an apparatus causes to the patient a local sensation of pain. The same problems arise in apparatuses for extraction of samples of body tissues.

It is therefore an object of the present invention to provide an apparatus which solves the above mentioned problems while it also reduces the duration of the treatment.

### SUMMARY

This object is solved according to the independent claims. Advantageous examples and embodiments are mentioned in the depending claims.

In a first aspect of the invention a medical device for infusion of an active ingredient may comprise a outlet device for infusion of an active ingredient to a body tissue; a receptacle for storing the active ingredient to which the outlet device is attachable; a sensor device for measuring a parameter which is related to a physical state in the body tissue and a drive device for effectuating at least an infusion of the active ingredient. Furthermore a control unit functionally connected to the sensor device for receiving a sensor signal, which corresponds to the state of the body tissue, wherein the control unit is adapted for controlling a flow rate of the active ingredient based on a signal received from the sensor device by commanding of the drive device, may be provided.

In a second aspect of the invention a medical device for removal of body tissue may comprise a outlet device for receiving the body tissue; a receptacle for storing the body tissue to which the outlet device is attachable; a sensor device for measuring a parameter which is related to a physical state in the body tissue and a drive device for effectuating at least a removal of the body tissue. Furthermore a control unit functionally connected to the sensor device for receiving a sensor signal, which corresponds to the state of the body tissue, wherein the control unit is adapted for controlling a flow rate of the body tissue removal based on a signal received from the sensor device by commanding of the drive device, may be provided.

According to a further embodiment of the first and second aspect of the present invention, the control unit is adapted to control the infusion of the active ingredient or the body tissue removal such that a measured parameter in the body tissue remains below a predetermined value in the body tissue during the infusion of the active ingredient or the removal of the body tissue.

Preferably, a function which derives the predetermined value from a total amount of active ingredient to be infused to the body tissue or a total amount of the body tissue to be removed from the body tissue over a predetermined time period may be provided.

Furthermore, the sensor device may be a pressure sensor adapted for measuring the pressure in the body tissue and/or the sensor device may be a shear stress sensor adapted for measuring the shear stress in the body tissue caused by the infusion of the active ingredient and/or by the removal of the body tissue.

In a third aspect of the invention a medical device for infusion of an active ingredient may comprise a outlet device for applying an active ingredient to a body tissue; a receptacle for storing the active ingredient to which the outlet device is attachable and a drive device for effectuating at least an infusion of the active ingredient. Furthermore a control unit functionally connected to the sensor device may be provided being adapted to control the infusion of the active ingredient according to a predetermined infusion process which is defined by a profile of an amount of active ingredient infused during a time period.

In a forth aspect of the invention a medical device for removal of a body tissue may comprise a outlet device for receiving the body tissue to be removed; a receptacle for storing the removed body tissue to which the outlet device is attachable and a drive device for effectuating at least a removal of the body tissue. Furthermore, a control unit functionally connected to the sensor device may be provided being adapted to control the removal of the body tissue according to a predetermined removal process which is defined by a profile of an amount of body tissue to be removed during a time period.

In a further embodiment of the first and second aspect of the invention the medical device may further comprise an input unit for inputting patient specific data, wherein the predetermined value in the tissue depends on the patient specific data.

In an embodiment of the third and forth aspect of the invention the medical device may further comprising an input unit for inputting patient specific data, wherein the profile depends on the patient specific data.

Preferably, the medical device may comprise a storing device for storing the specific patient data in a data set.

Furthermore, a storing device for storing the data set and a command function which derives a flow rate profile from the data set, depending on the patient specific data may be provided.

Additionally, the patient data may comprise at least the age, the sex and/or known diseases.

Furthermore, an air vessel connected to the drive device and adapted for creating a flow rate during the infusion of the active ingredient and/or the removal of the body tissue may be provided.

The above mentioned features may be combined in any way.

### BRIEF DESCRIPTION OF THE FIGURES

Preferred embodiments of the invention will now be described based on the accompanying drawings, wherein

FIG. 1 shows a functional scheme of the medical device according to a first aspect of the invention, and

FIG. 2 shows a functional scheme of the medical device according to a second aspect of the invention.

### DETAILED DESCRIPTION

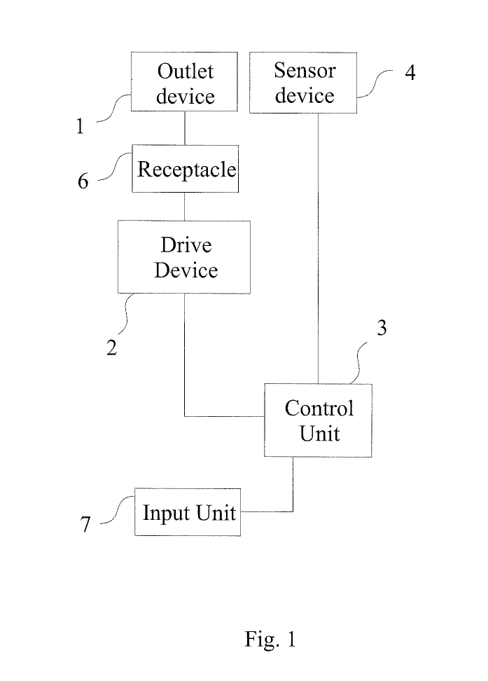

FIG. 1 shows a medical device, in particular a portable medical device, comprising an outlet device 1 for infusion of an active ingredient to a body tissue; a receptacle 6 for storing the active ingredient to which the outlet device 1 is attachable; a sensor device 4 for measuring a parameter which is related to a physical state in the body tissue ; a drive device 2 for effectuating at least an infusion of the active ingredient. The control unit 3 is functionally connected to the sensor device 4 for receiving a sensor signal, which corresponds to the state of the body tissue. The outlet device 1 may be in particular a needle. The sensor device 1 measures a parameter which is related to the state of a body tissue in which a medicament is infused, or from which a part of body tissue is removed. The sensor device 4 outputs a sensor signal corresponding to the measurement of the sensor device.

This sensor signal is then input to the control unit 3. Furthermore, the control unit 3 can also receive a signal from the input unit 7. The signal from the input unit 7 contains a data set comprising patient specific data like for instance the age, the sex, the weight and/or known diseases. This data is input by a user via the input unit 7.

The control unit 3 outputs a command signal which depends on the received signals. The command signal is then input to the drive device 2. The drive device 2 is adapted to operate a piston means moveably disposed in a receptacle 6 which comprises a medicament. For those skilled in the art it will be clear that such an operation will be performed by a plunger or a piston which protrudes into the receptacle such that the medicament is then output via the outlet device 1.

In an embodiment of the invention, the sensor device 4 comprises a pressure sensor for sensing the pressure in the body tissue of a patient near the penetration point of the outlet device 1. During the surgery of a patient, the control unit 3 receives the sensor signal corresponding to a pressure value in the body tissue. Then the control unit 3 compares the measured value with a predetermined value that corresponds to a maximum allowable value of pressure in the tissue. If the measured value exceeds or equals the maximum predetermined value or a value derived therefrom the control unit 3 outputs a command signal that causes the drive device 2 to reduce the flow rate of the medicament which flows from the receptacle 6 via the outlet device 1 into the tissue. Therefore the pressure in the body tissue can be kept under a certain level, such that the sensation of pain of a patient can be systematically reduced.

According to a second aspect of the invention the medical device shown in FIG. 1 can also be used for the removal of body tissue from a human being. According to an example of the invention the sensor device 4 is adapted such that it measures the shear stress in the body tissue or the body liquid. In an alternative embodiment the sensor device 4 may be adapted such that it measures the pressure in the body tissue or the body liquid. During for instance a biopsy, the outlet device 1 is introduced into an area of patient's body tissue. Then the drive device 2 is caused to remove a body tissue which is introduced in an inside portion of the outlet device 1 to be transferred to the receptacle 6. This may be carried out especially by creation of a under pressure in the receptacle 6 with the drive device 2 in the receptacle 6.

At the same time the sensor device 4 measures the shear stress in the body tissue area from which the sample is removed and transmits a sensor signal to the control unit 3. The control unit 3 receives this signal and compares the value of the measured shear stress with a maximum allowable shear stress. As mentioned above, if the control unit 3 detects, that the measured value exceeds a predetermined maximum value it outputs a command value to the drive device 2. Accordingly, the drive device 2 is controlled such that a reduction of the flow rate is performed in order to reduce the shear stress in the body tissue. Accordingly, with such an arrangement the pain sensation of the patient can be reduced.

With a parametrical control according to the invention the flow rate can be controlled depending on parameters as for instance time, pressure in the body tissue infusion volume and shear stress. This parametrical control comprises the measurement of a parameter value, for example the pressure or the shear stress in the tissue, the comparison of the measured parameter value with a predetermined parameter value, for instance a predetermined maximum pressure value and the command of a drive device, for example an electromechanical pump for correcting the real pressure in the body tissue. Furthermore, a parametrical control of the removal of body tissue can also be performed with a device according to the invention, wherein fluidic, gaseous volumes as for instance blood, bone marrow, biopsies or drainage fluids can be collected in the receptacle 6.

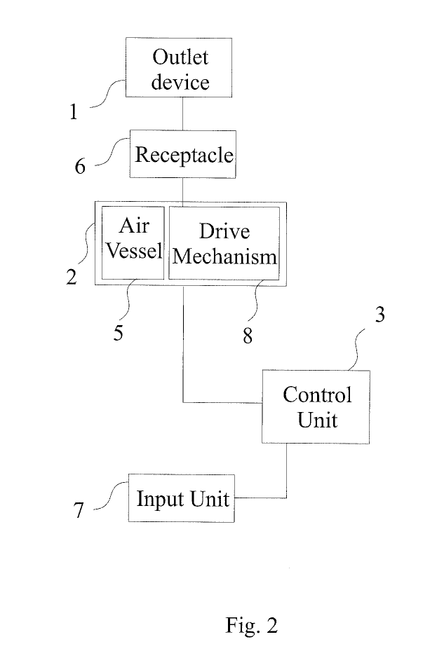

In FIG. 2 a further embodiment of the present invention is shown, wherein the medical device for infusion of an active ingredient comprises an outlet device 1 for infusion of an active ingredient to a body tissue; a receptacle 6 for storing the active ingredient to which the outlet device 1 is attachable; a drive device 2 for effectuating at least an infusion of the active ingredient. The control unit 3 is functionally connected to the sensor device 4 being adapted to control the infusion of the active ingredient according to a predetermined infusion process which is defined by a time dependent profile of an amount of active ingredient infused during a time period.

The actuator 5 is embodied as an air vessel, however, for those skilled in the art it is obvious that various other examples for the actuator can be foreseen. The example given in FIG. 2 functions based on the so called windkessel-effect. Thereby, an amount of air is set under pressure beforehand wherein the air pressure is transferred to the receptacle 6. Accordingly, the pressure in the receptacle 6 follows each variation of the air pressure in the air vessel. In the case when the medical device is used for infusion of a medicament the effect is that at the beginning of the infusion a high pressure is present in the air and therefore also in the medicament. During the output of the medicament the pressure in the air decreases as also does the pressure in the receptacle 6 and accordingly the pressure in the body tissue. With such an arrangement it is possible to create a profile of the flow rate over a time period in a very simple way.

The control unit 3 outputs a command signal which is input to the drive device 2 so that the air vessel 5 is configured with a predetermined pressure respectively. In FIG. 2 an input unit 7 is also provided with which a user inputs patient specific data as mentioned above. The command signal output by the control unit is dependent on this input data which it receives from the input unit 7.

The schema shown in FIG. 2 also applies for a medical device which is adapted for removal of body tissue from a patient. When the medical device is used therefore, an under pressure is created in the air vessel, such that an under pressure is also created in the receptacle leading to a suction effect on the body tissue. Thereby the body tissue is sucked in the receptacle 6.

Besides the use for insulin application the present invention may also be applied to biomarker or bio-feed back controlled therapy. Stabilisation of a heart function with substances is an alternative use of the invention. Under detection of the heart frequency the infusion of the substance can be permanently controlled. As a further example the survey of a narcosis shall be mentioned. Usually pathologic states are surveyed by an anaesthetist and when a measured value reaches a predetermined value an alert signal is output. The correction of the application is then carried out manually. The present invention therefore provides the advantage, that the biomarker control can be carried out automatically.

According to the above described embodiments a parametric control of the infusion velocity depending on the time or the infusion pressure can be achieved. Therefore an undesired, excessive increase of the pressure in the tissue leading to pain sensation can be inhibited. This is performed by control of the pressure in the tissue by measurement of the pressure in the tissue or via a time depending flow rate profile which results in a time depending pressure profile.

Furthermore a dependency of the shear stress can be realised during a process of removal of the body tissue. The vitality of bone marrow cells after their extraction can be increased. A further advantage of the invention lies therein, that smaller samples can be extracted leading to a reduction of the surgery duration. Therefore a standardisation can facilitated.

1. A medical device for infusion of an active ingredient comprising
a outlet device for infusion of an active ingredient to a body tissue;
a receptacle for storing the active ingredient to which the outlet device is attachable;
a sensor device for measuring a parameter which is related to a physical state in the body tissue;
a drive device for effectuating at least an infusion of the active ingredient;
characterized by
a control unit functionally connected to the sensor device for receiving a sensor signal, which corresponds to the state of the body tissue, wherein the control unit is adapted for controlling a flow rate of the active ingredient based on a signal received from the sensor device by commanding the drive device.

2. A medical device for removal of body tissue or liquid comprising
a outlet device for receiving the body tissue or liquid;
a receptacle for storing the body tissue or liquid to which the outlet device is attachable;
a sensor device for measuring a parameter which is related to a physical state in the body tissue ;
a drive device for effectuating at least a removal of the body tissue or liquid;
characterized by
a control unit functionally connected to the sensor device for receiving a sensor signal, which corresponds to the state of the body tissue or liquid, wherein the control unit is adapted for controlling a flow rate of the body tissue or liquid being removed based on a signal received from the sensor device by commanding of the drive device.

3. The device according to claim 1, wherein the control unit is adapted to control the infusion of the active ingredient or the body tissue being removed such that a measured parameter in the body tissue or liquid remains below a predetermined value in the body tissue during the infusion of the active ingredient or the removal of the body tissue or liquid.

4. The device according to claim 3, further comprising a function which derives the predetermined value from a total amount of active ingredient to be infused to the body tissue or a total amount of the body tissue or liquid to be removed from the body tissue over a predetermined time period.

5. The device according to claim 1, wherein the sensor device is a pressure sensor adapted for measuring the pressure in the body tissue and/or the sensor device is a shear stress sensor adapted for measuring the shear stress in the body tissue caused by the infusion of the active ingredient and/or by the removal of the body tissue or liquid.

6. A medical device for infusion of an active ingredient comprising:
a outlet device for infusion of an active ingredient to a body tissue;
a receptacle for storing the active ingredient to which the outlet device is attachable;
a drive device for effectuating at least an infusion of the active ingredient;
characterized by
a control unit functionally connected to the sensor device being adapted to control the infusion of the active ingredient according to a predetermined infusion process which is defined by a time dependent profile of an amount of active ingredient infused during a time period.

7. A medical device for removal of a body tissue or liquid comprising;
a outlet device for receiving the body tissue or liquid to be removed;
a receptacle for storing the removed body tissue or liquid to which the outlet device is attachable;
a drive device for effectuating at least a removal of the body tissue or liquid;
characterized by
a control unit functionally connected to the sensor device being adapted to control the removal of the body tissue or liquid according to a predetermined removal process which is defined by a profile of an amount of body tissue or liquid to be removed during a time period.

8. A device according to claim 1, further comprising an input unit for inputting patient specific data, wherein the predetermined value in the tissue depends on the patient specific data.

9. A device according to claim 6, further comprising an input unit for inputting patient specific data, wherein the profile depends on the patient specific data.

10. A device according to claim 8, comprising a storing device for storing the specific patient data in a data set.

11. A device according to claim 6, comprising a storing device for storing the data set and a command function which derives a flow rate profile from the data set, depending on the patient specific data.

12. A device according to claim 8, wherein the patient data comprises at least the age, the sex and/or known diseases.

13. A device according to one of claim 6, further comprising an air vessel connected to the drive device and adapted for creating a flow rate during the infusion of the active ingredient and/or the removal of the body tissue or liquid.
